(19) United States
(12) Patent Application Publication
(10) Pub. No.: US 2008/0199370 A1
(43) Pub. Date: Aug. 21, 2008

(54) Efficient infrared-based reaction vessel

(76) Inventors: Firas Mourtada, Pearland, TX (US); Ali Azhdarinia, Friendswood, TX (US); David Yang, Sugar Land, TX (US); Christian Landry, Sugar Land, TX (US)

Correspondence Address: VINSON & ELKINS L.L.P., First City Tower, 1001 Fannin Street, Suite 2300, HOUSTON, TX, 77002-6760, US

(21) Appl. No.: 12/004,340

(22) Filed: Dec. 20, 2007

Related U.S. Application Data
(60) Provisional application No. 60/876,039, filed on Dec. 20, 2006.

(51) Int. Cl. A61K 51/00; B01J 19/12

(52) U.S. Cl. 422/186.04; 422/186; 424/1.11

(57) ABSTRACT

The present invention provides an efficient infrared (IR) reaction vessel system that includes an infrared reaction vessel having an optical window into a reaction reservoir that absorbs minimal infrared energy, a male cap attached to the reaction vessel that allows precise positioning with respect to an infrared heat source, and at least one access tube passing through the cap. The infrared reaction vessel of the present invention further provides vacuum lines that also repulse ions and minimize evaporation loss and novel temperature control feedbacks such as the use of a proximally positioned resistive thermal device (RTD).

### CROSS REFERENCE TO RELATED APPLICATION

This application claims priority to U.S. Provisional application No. 60/876,039, filed Dec. 20, 2006.

### FIELD OF THE INVENTION

The present invention relates to the field of radiopharmaceutical synthesizers. More particularly, the invention relates to an improved apparatus for, and method of, heating reactions during the production of radiochemistry formulations.

### DESCRIPTION OF RELATED ART

Non-invasive medical imaging techniques such as Positron Emission Tomography (PET) and Single Photon Emission Computed Tomography (SPECT) have been experiencing explosive growth due to advances in functional imaging technology. New molecular imaging targets for diagnosis and therapy have been developed to visualize disease states and pathological processes without surgical exploration of the body. In particular, targeted radiopharmaceuticals offer promising capabilities for the non-invasive assessment of the pathophysiology of diseases. Schillaci, O. & Simonetti, G., Cancer Biother. Radiopharm. 19: 1-10 (2004); Paulino, et al, Semin. Nucl. Med. 33: 238-43 (2003). However, radiopharmaceuticals suitable for clinical use have been limited, which has led to the recent development of new radiopharmaceuticals with improved sensitivity, specificity, signal-to-background ratio and biodistribution. Srivastava, S. C., Semin. Nucl. Med. 26: 119-31 (1996); Gatley, et al, Acta. Radiol. Suppl. 374: 7-11 (1990); Mason, N. S. & Mathis, C. A., Neuroimaging Clin. N. Am. 13, 671-87 (2003).

Nuclear imaging consists of chemicals or biochemicals that are tagged with radioactive materials to provide contrast between sites which take up the agent and those which do not. Development of such agents relies on a variety of radiochemistry techniques, which are performed by trained radiochemists. One factor, however, that has limited the number of suitable radiopharmaceuticals available relates to the relatively short half-lives of the radioisotopes used in radiopharmaceuticals. Short half-lives are required to provide a strong signal during imaging and to subsequently limit the patient's exposure to radioactive materials after the imaging is completed. On the other hand, however, the continuous decay of radioisotopes also make large scale production and storage for any extended period of time impracticable. Therefore, most radiochemists perform a manual or automated syntheses of imaging radiotracers just prior to use.

Radioisotopes are often created by a cyclotron or via a generator-based synthesis protocol. Cyclotrons are large and costly systems, and as a result, many medical imaging facilities must obtain their radioisotopes from cyclotron facilities that are significant distances away. The time that it takes to synthesize a radiopharmaceutical, and then deliver it to a medical imaging facility may necessitate that radioisotopes require a larger quantity or higher activity.

Alternatively, investigators can use generators based on a parent-daughter (P/D) nuclidic pairing, wherein a relatively long-lived parent isotope (for example, obtained from a cyclotron) decays to a short-lived daughter isotope better suited for imaging. The parent isotope can be shipped to a clinical site and act as the source from which the daughter isotope can be readily eluted. Generators of this type are generally smaller and relatively inexpensive and therefore are more readily affordable for use on-site at medical imaging facilities.

Numerous types of generator systems are known to those skilled in the art and any generator system that produces a sufficient quantity of a daughter nuclide can be useful in medical imaging or therapeutics including, but not limited to parent/daughter pairs: ⁶⁸Germanium and ⁶⁸Gallium, ⁴⁴Titanium and ⁴⁴Scandium, ⁵²Iron and ^{52m}Manganese, ⁶²Zinc and ⁶²Copper, ⁷²Selenium and ⁷²Arsenic, ⁸²Strontium and ⁸²Rubidium, ⁹⁹Molybdenum and ^{99m}Technetium, ¹¹⁸Tellurium and ¹¹⁸Antimony, ¹²²Xenon and ¹²²Iodine, ¹²⁸Barium and ¹²⁸Cesium, ¹⁸⁸Tungsten and ¹⁸⁸Uranium, ¹⁷⁸Tungsten and talum, and ^{195m}Mercury and ^{195m}Gold.

During radioisotope synthesis and complexation, reproducibility, reaction time and radioactive exposure are key concerns. Radioisotope purification and radiopharmaceutical formulation require intricate handling of radioactive materials, fast reaction times, ease of synthesis and reproducible results. Therefore, radiotracers undergo detailed quality control analysis to assess the following: (1) the synthesized radiopharmaceuticals must meet strict sterility and pyrogenicity requirements which must be validated from batch to batch; (2) the system must be highly reproducible from batch to batch, demonstrating suitable radiochemical yield, radiochemical purity, pH and specific activity; (3) the synthesis time must be fast when dealing with radionuclides with a short half-life or the nuclides will lose their utility as radiotracers; and (4) the purification and synthesis equipment and protocols used must afford maximal protection for radiochemists doing the purification and synthesis by minimizing their exposure to the highly radioactive materials being handled.

To address these requirements, automated synthesizers have been developed over the last two decades for radiopharmaceutical synthesis. For example, the GE TRACERLAB® MX line of products are fully-automated systems for radiopharmaceutical synthesis have been developed for synthesis of radiopharmaceuticals from cyclotron-derived radioisotopes. Meyer et al. discloses a semi-automated system for purification of generator-based ⁶⁸Ga and synthesis of a single type of ⁶⁸Ga radiopharmaceutical, DOTA-derivatized peptide ligands. Meyer et al. Eur. J. Nucl. Med. Mol. Imaging, 31:1097-1104 (2004). Furthermore, WO 2005/057589 discloses systems and methods for synthesizing oil-soluble and water-soluble radioisotopic agents and provides automated systems for preparing radioisotopes and subsequently synthesizing radiotracers from those isotopes.

Two commercially available automated synthesizer for generator-based PET imaging are available. One unit incorporates the ⁶²Cu generator (⁶²Zn⁶²Cu). The second unit is a modular lab for synthesis of ⁶⁸Ga-based agents. Similarly, U.S. Patent Application Ser. No. 60/822,306, which is hereby incorporated by reference, discloses an automated system for the creating generator based radiopharmaceuticals suitable for use in a variety of imaging procedures and is not limited to a single parent-daughter nuclidic pairings or particular complexing reaction. This system utilizes a closed, or partially closed, network of tubing in fluid communication with various reaction, measurement, and purification stations to produce radiopharmaceuticals.

Regardless of the radioisotope source, the specific radiochemistry formulations, or whether the process is manually performed or utilizes an automated system, the involved reactions often require heating to complete at least a one-reaction step. Conventional heating methods include convection- and conduction-based heating in oil baths, resistively heated metal blocks, and hot air guns used to heat liquids in reaction vessels. Nevertheless, because convection and conduction are indirect heating methods (i.e., both rely on the transmission or propagation of heat through some intermediate physical medium) both are prone to longer heating times and larger variations from targeted temperatures. These two factors can drastically affect both reaction time and yield during radiopharmaceutical synthesis.

Microwave based heating systems have been designed to overcome some of the drawbacks associated with more conventional heating methods. See Velikyan et al., “Microwave-Supported Preparation of ⁶⁸Ga Bioconjugates with High Specific Radioactivity,” Bioconjugate Chem. 15:554-560 (2004). When subjected to microwave based heating a sample is heated directly, as opposed to a heat being applied to a reaction vessel wall and then conducted into the sample. This results in a more uniform and quicker heating of the sample.

Optical based energy systems such as lasers have also been developed for use in heating chemical reactions in an attempt to overcome some of these conventional heating drawbacks. For example, U.S. Pat. No. 4,025,408 discloses the use of coherent energy from a laser to induce a reaction by exciting a specific molecular bond in a reactant. Another optical based heating system is disclosed in U.S. Pat. No. 4,257,860 that utilizes an infrared (IR) laser to dissociate a compound for use in deuterium enrichment.

Infrared-based mechanisms provide an attractive source of heat for radiopharmaceutical synthesis because of increased heating efficiency and the commercial availability of multiple types and configurations of IR heaters and the fact that infrared energy can be absorbed directly by a suitable reaction medium. U.S. Pat. No. 5,808,020 describes an optical reaction cell or vessel for utilizing infrared heat during ¹⁸Fluorine-FDG synthesis and is hereby incorporated by reference. The reaction vessel described includes a reaction reservoir that is substantially inert to the contents of a reaction medium, an optical window that is substantially transmissive to infrared radiant energy and preferably includes an infrared-reflecting surface that is highly reflective to infrared radiant energy passing through the reaction volume to redirect infrared radiant energy passing through the reaction reservoir back into the reaction reservoir.

Nevertheless, infrared heating of chemical reactions, especially those involved in radiopharmaceutical synthesis still have several drawbacks. For example, precise temperature measurements and control can be difficult to obtain, especially in a closed-loop system such as those typically used in automated systems. Furthermore, contamination concerns often necessitate the use of disposable, or at a minimum removable, infrared reaction vessels. Variations in location within such systems with respect to the position relative to an infrared source will directly affect reaction time and yield. Finally, the manual. manipulations of reactions required in radiopharmaceutical development, such as the addition of reactants, agitation of reactants, and removal of reactants can be less precise and reproducible as compared to automated, or semi-automated IR-based heating systems.

Therefore there is a need in the art for an infrared reaction vessel that is reproducibly positionable with respect to an infrared source, and allows for access to the reaction volume in a closed or semi-closed loop system.

### BRIEF SUMMARY OF THE INVENTION

The present invention provides an infrared reaction vessel system and method of use therefore that include: a reaction reservoir formed by the walls of a reaction vessel; an optical window into the reaction reservoir that absorbs minimal infrared energy; a male cap attached to the reaction vessel; and at least one access tube passing through the cap. In certain embodiments, the infrared reaction vessel also includes an associated support bracket, wherein the support bracket remains at a fixed location relative to an infrared source; a male key cap which fits into a female receptacle located on the support bracket in only one position; and a vacuum line connected to at least one of the access tubes. Certain embodiments of the vacuum lines also repulse ions and minimize evaporation loss. The invention further includes novel temperature control feedbacks such as the use of a proximally positioned resistive thermal device (RTD).

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Diagram of one embodiment of a circular IR reaction vessel with a support bracket.

FIG. 2A: Diagram of an IR reaction vessel positioned in front of an IR heater (focal-spot type).

FIG. 2B: Diagram of an IR reaction vessel positioned in front of an IR heater (strip type with rectangular planar profile).

FIG. 3A: Schematic of a circular heat profile projecting from an IR focal-type source.

FIG. 3B: Schematic of a rectangular heat profile projecting from an IR strip-type source.

FIG. 4A: Front angle view of an embodiment of a rectangular IR reaction vessel.

FIG. 4B: Side view of an embodiment of a rectangular IR reaction vessel.

FIG. 4C: Diagram of an IR reaction vessel positioned in front of an IR heater (strip type).

FIG. 5: Cross-sectional view of a circular IR reaction vessel.

FIG. 6A: Multiple views of a vacuum line in a loop-configuration with a condensation trap.

FIG. 6B: Multiple views of a vacuum line in a loop-configuration with a mesh evaporation guard.

FIG. 7: Schematic of an automated radiopharmaceutical synthesis platform with a removable IR reaction vessel.

### DETAILED DESCRIPTION OF THE INVENTION

For the purposes of the present invention “radiopharmaceuticals” are compounds suitable for use in medical applications such as nuclear imaging, chemotherapy and the like. Radiopharmaceuticals are generally provided in a pharmaceutically-acceptable carrier.

As used herein “formulation” of a radiopharmaceutical preferably means chemically reacting a radioisotope with a ligand to produce a compound suitable for use as a radiopharmaceutical, but additionally can mean adjusting the pH, concentration or other physical characteristics of a radiopharmaceutical preparation to render it suitable for pharmaceutical use.

The present invention provides an infrared (IR) reaction vessel and both automated and manual methods for the use thereof. The present invention includes several novel features added to a reaction vessel coupled to an IR heater to maximize absorbance of IR energy thus reducing synthesis reaction time. Also, the IR reaction vessel of the present invention furthermore is designed to improve heat profile reproducibility between reactions, to reduce radioactivity loss in vacuum lines used to vent reactions, and to provide a disposable or replaceable unit with seamless installation between runs.

The invented vessel can be incorporated into radiopharmaceutical synthesis systems or protocols utilizing infrared-based heating, in particular for PET imaging with generator based tracers (based on a parent-daughter nuclidic pair such as a ⁶⁸Ga, ⁴⁴Sc, ^{52m}Mn, ⁶²Cu, ⁷²As, ⁸²Rb, ^{99m}Tc, ¹¹⁸Sb, ¹²²I, ¹²⁸Cs, ¹⁷⁸Ta or ^{195m}Au generators), cyclotron-based radiotracers (¹⁸F, ⁶¹Cu, ⁶⁴Cu, etc.), as well as for therapy-based agents like ¹⁸⁸Re.

The apparatus and methods of the present invention are, furthermore, useful in a number of different radioisotope production protocols and a number of different radiopharmaceutical production protocols, including radiopharmaceuticals that have a bioconjugate, a radioisotope(s), and a targeting molecule. Examples of suitable bioconjugates known to those skilled in the art to be useful in complexing to radioisotopes includes, but is not limited to, desferal-based bioconjugates, bifunctional chelators based on tetraazo compounds such as 1,4,7-triazacyclonane-N,N′,N″-tri-acetic acid and 1,4,7,10-tetraazacyclododecane-N,N′,N″,N′″-tetraacetic acid (DOTA) and cyclams, (1-(1-carboxy-3-carboxy-propyl)-4,7-(carboxy,methyl)-1,4,7-triazacyclononane (NODAGA), diethylenetriaminepenaacetic acid (DTPA), hydrazinonicotinamide (HYNIC), mercaptoacetyltriglycine (MAG3), ethylenedicysteine (EC), Tyr³-octreotide based bioconjugates such as DOTA⁰-D-Phe¹-Tyr³-octreotide (DOTATOC) and NODAGA-Tyr3-octreotide (NODAGATOC), S₃N ligands such as bis(2-(benzylthio)benzyl)(2-(benzylthio)4-aminobenzyl)amine, and numerous others that will be apparent to those skilled in the art.

Suitable bioconjugates generally serve two main purposes: 1) to coordinate the radiometal and 2) to provide a molecular backbone that can be modified with functional groups for attachment to the targeting molecule. Conjugation of radiometal chelators can be applied to multiple classes of compounds described below.

Classes of targeting molecules include, but are not limited to, disease cell cycle targeting compounds, angiogenesis targeting ligands, tumor apoptosis targeting ligands, disease receptor targeting ligands, drug-based ligands, antimicrobials, agents that mimic-glucose, tumor hypoxia targeting ligands and the like.

Disease cell cycle targeting compounds are often nucleoside analogues. For example, pyrimidine nucleoside (e.g., 2′-fluoro-2′-deoxy-5-iodo-1-β-D-arabinofuranosyluracil (FIAU), 2′-fluoro-2′-deoxy-5-iodo-1-β-D-ribofuranosyl-uracil (FIRU), 2′-fluoro-2′-5-methyl-1-β-D-arabinofuranosyluracil (FMAU), 2′-fluoro-2′-deoxy-5-iodovinyl-1-β-D-ribofuranosyluracil (IVFRU) and acycloguanosine: 9-[(2-hydroxy-1-(hydroxymethyl)ethoxy)methyl]guanine (GCV) and 9-[4-hydroxy-3-(hydroxymethyl)butyl]guanine (PCV) and other 18F-labeled acycloguanosine analogs, such as 8-fluoro-9-[(2-hydroxy-1-(hydroxymethyl)ethoxy)methyl]guanine (FGCV), 8-fluoro-9-[4-hydroxy-3-(hydroxymethyl)butyl]guanine (FPCV), 9-[3-fluoro-1-hydroxy-2-propoxy methyl]guanine (FHPG) and 9-[4-fluoro-3-(hydroxymethyl)butyl]guanine (FHBG) have been developed as reporter substrates for imaging wild-type and mutant HSV1-tk expression.

Examples of angiogenesis targeting ligands include COX-2 inhibitors, anti-EGF receptor ligands, herceptin, angiostatin, C225, and thalidomide. COX-2 inhibitors include, for example, celecoxib, rofecoxib, etoricoxib, and analogs of these agents.

Tumor apoptosis targeting ligands include, but are not limited to, TRAIL (TNF-related apoptosis inducing ligand) monoclonal antibody, substrates of caspase-3, such as peptide or polypeptide that includes the 4 amino acid sequence aspartic acid-glutamic acid-valine-aspartic acid, any member of the Bcl family.

Examples of disease receptor targeting ligands include, but are not limited to, estrogen receptors, androgen receptors, pituitary receptors, transferrin receptors, and progesterone receptors. Examples of agents that can be applied in disease-receptor targeting include androgen, estrogen, somatostatin, progesterone, transferrin, luteinizing hormone, and luteinizing hormone antibody. The folate receptor is included herein as another example of a disease receptor. Examples of folate receptor targeting ligands include folic acid and analogs of folic acid. Preferred folate receptor targeting ligands include folate, methotrexate and tomudex.

Certain drug-based ligands can be applied in measuring the pharmacological response of a subject to a drug. A wide range of parameters can be measured in determining the response of a subject to administration of a drug. One of ordinary skill in the art would be familiar with the types of responses that can be measured. These responses depend in part upon various factors, including the particular drug that is being evaluated, the particular disease or condition for which the subject is being treated, and characteristics of the subject. Examples of drug-based ligands include carnitine and puromycin.

Any antimicrobial is contemplated for inclusion as a targeting ligand. Preferred antimicrobials include ampicillin, amoxicillin, penicillin, cephalosporin, clidamycin, gentamycin, kanamycin, neomycin, natamycin, nafcillin, rifampin, tetracyclin, vancomycin, bleomycin, and doxycyclin for gram positive and negative bacteria and amphotericin B, amantadine, nystatin, ketoconazole, polymycin, acyclovir, and ganciclovir for fungi.

Agents that mimic glucose are also contemplated for inclusion as targeting ligands. Preferred agents that mimic glucose, or sugars, include neomycin, kanamycin, gentamycin, paromycin, amikacin, tobramycin, netilmicin, ribostamycin, sisomicin, micromicin, lividomycin, dibekacin, isepamicin, astromicin, aminoglycosides, glucose or glucosamine.

Tumor hypoxia targeting ligands are also useful in certain embodiments of the present invention. Misonidazole, an example of a tumor hypoxia targeting ligand, is a hypoxic cell sensitizer, and labeling MISO with different radioisotopes (e.g., ¹⁸F, ¹²³I, ^{99m}Tc) may be useful for differentiating a hypoxic but metabolically active tumor from a well oxygenated active tumor by PET or planar scintigraphy. [¹⁸F]Fluoromisonidazole (FMISO) has been used with PET to evaluate tumor hypoxia.

Other suitable reagents and protocols for the formulation of radiopharmacueticals will be apparent to those skilled in the art and may be readily adapted for use with the apparatus and methods of the present invention.

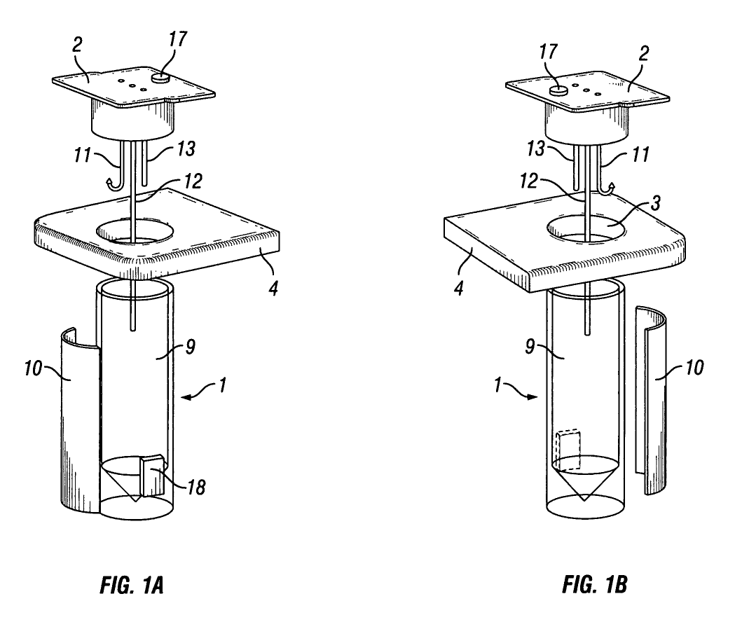

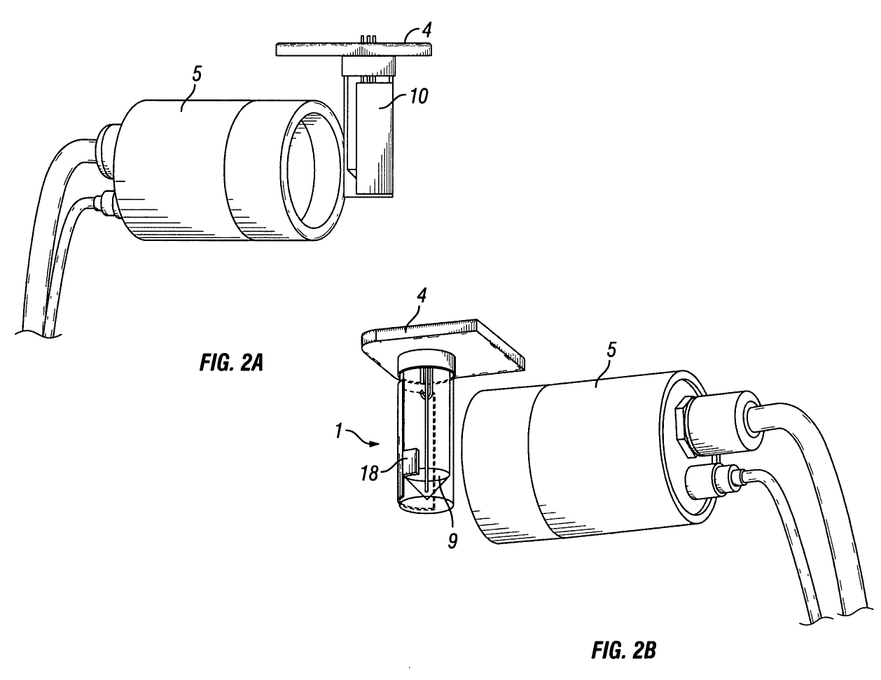

Any reaction requiring heat to initiate or sustain the reaction in the formulation of a radiopharmaceutical is suitable candidate for performing in the IR reaction vessel of the present invention. FIG. 1 depicts one embodiment of the IR reaction vessel 1 of the present invention. In this particular embodiment, the reaction vessel 1 is a modified version of a common 2-10 ml conical unleachable borosilicate vial, or similar vial. The reaction vessel connects to a male key cap 2 which is formed in a geometric shape that will fit into a female key component 3 located on a support bracket 4. The support bracket 4 is in a fixed location with respect to an IR source 5 (whether the bracket is used in a manual procedure or is part of an automated apparatus). As shown in FIGS. 2A, 2B, 4C and 7, the support bracket in certain embodiments can be permanently positioned in relationship to an IR source 5 such that, when the male key cap 2 is connected to the reaction vessel 1 and placed in the support bracket 4, the reaction reservoir 9 is positioned at a set, reproducible, location with respect to a given IR source 5. In certain embodiments, the male key cap 2 has a non-symmetrical two- or three-dimensional shape or configuration that will fit into the female key component 3 in only one position. One of ordinary skill in the art will recognize that the configuration shown in the Figures of the present disclosure are exemplary embodiments only. For example, the male and female aspects of the key cap and support bracket can be reversed to achieve the same functionality.

In certain embodiments, the IR reaction vessel 1 can be coded or designed such that it will only fit or function in an appropriate support bracket 4 on a specified automated or semi-automated platform. For example, the support bracket 4 can have a scanner associated with it that will not allow any process to proceed if a corresponding bar code is not read from the appropriate IR reaction vessel 1 after placement in the support bracket 4. In addition, certain embodiments are also coded to allow software to recognize the type of radiochemistry to be performed. Furthermore, certain IR reaction vessel 1 can be provided preloaded with appropriate reagents.

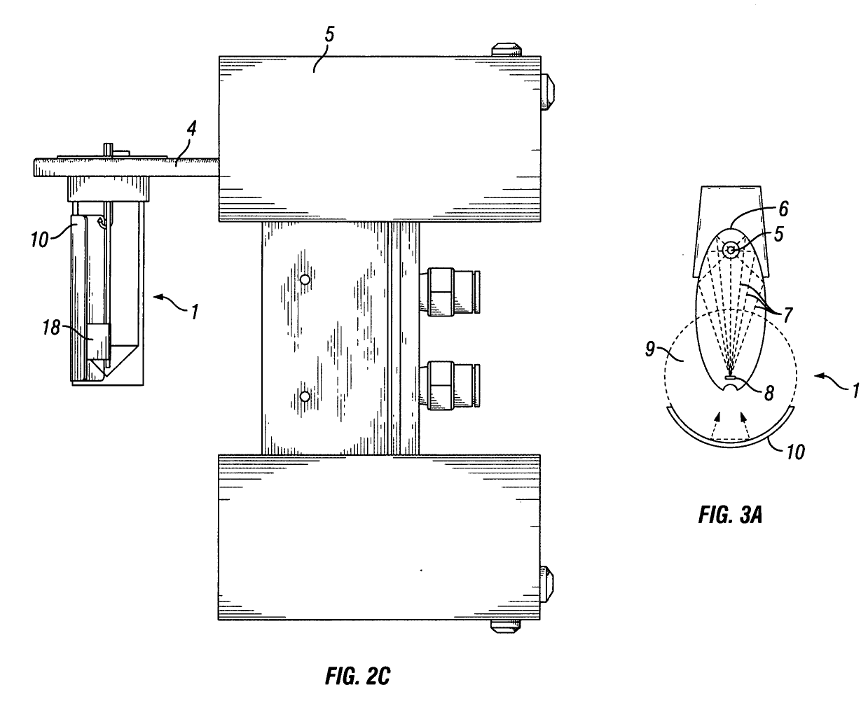

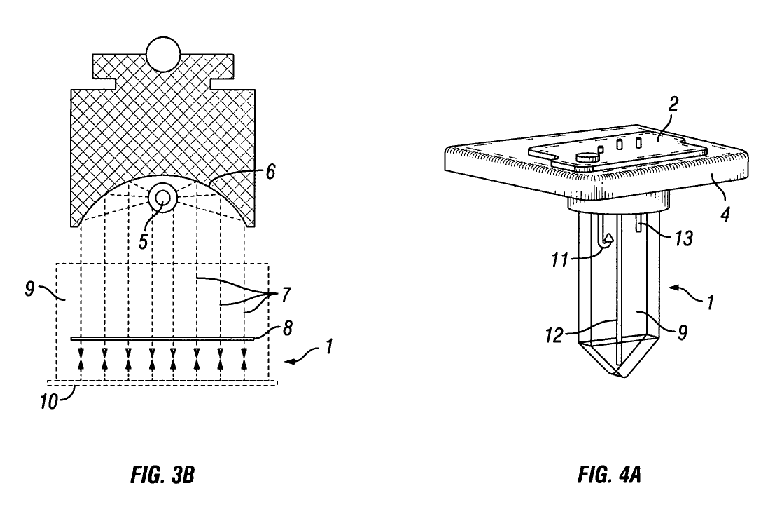

One of ordinary skill in the art will also readily recognize that the IR reaction vessel 1 can be configured in a variety of shapes and that different shapes can be matched with shape of IR source reflector 6 and resulting heat profile 8. As shown in FIG. 3A the shape of the reflector 6 behind the IR source 5 will determine the shape of the heat profile 7 projected toward the IR reaction vessel 1. In FIG. 3A, the IR source reflector 6 has an elliptical shape that focuses the heat profile 8 on a small circular location at a specific focal length from the IR source 5. This circular location is preferably positioned within the reaction reservoir 9 and within the volume of reactants contained therein. One of ordinary skill will recognize that the positioning of the circular location within the reaction reservoir 9 will directly effect heating times for the reactants and that such positioning can be readily optimized. In alternate embodiments, such as shown in FIG. 3B, an IR source reflector 6 can be configured to generate a more uniform and planar heat profile 8. In certain of these embodiments, the IR source 5 can be configured in a tubular or strip shape generating a heat profile 8 that is planar as shown in FIG. 3B but also having depth (i.e., projects as a cube or box shape) with inverse intensity. In such embodiments as with the embodiments utilizing an elliptical IR source shield 5, the positioning of the heat focal point, or plane 8, can be positioned to maximize heat distribution within the reaction reservoir 9, and thereby decrease heating time and/or volatility of solution during evaporation.

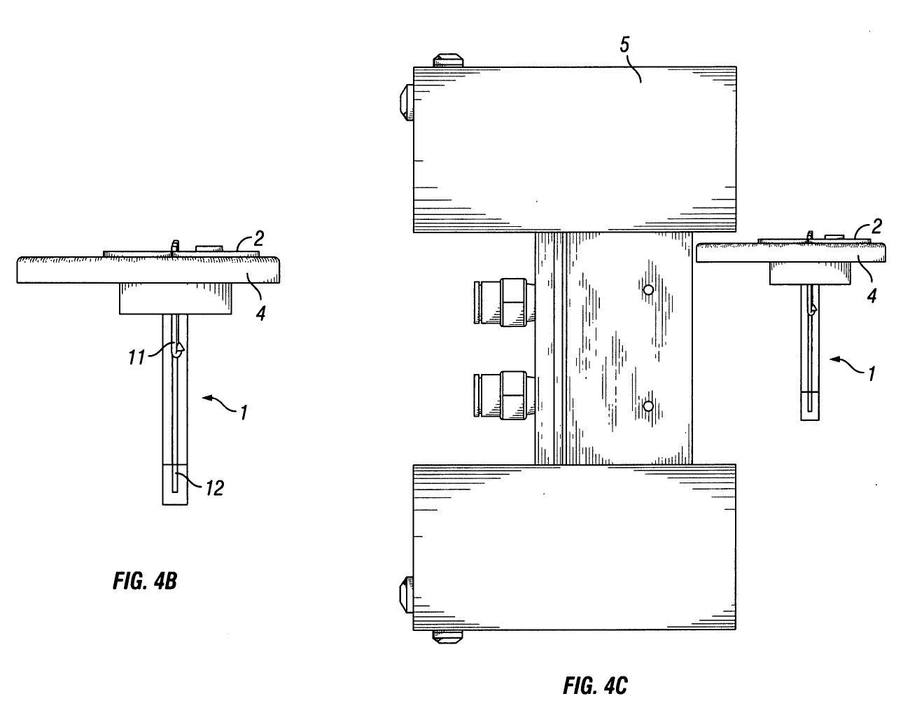

FIGS. 4A, 4B and 4C depict an embodiment of the IR reaction vessel 1 that is designed to increase the compatibility with the heat transfer profile displayed in FIG. 3B. In these embodiments, the IR reaction vessel has a rectangular shape to match the heat profile 8 and thereby maximize the heat distribution within the reaction reservoir 9. One of ordinary skill in the art will readily recognize that multiple IR sources 5 can also be employed to decrease heating time and increase the uniformity of the heat transfer profile 8.

The IR generated heat is transferred by electromagnetic waves of 0.78 to 1000 micron wavelengths. In use, however, not all IR is absorbed but rather some is transmitted beyond the volume of the reaction reservoir 9. In certain embodiments, the IR reaction vessel 1 also includes an IR reflective coating 10 order to redirect non-absorbed IR back into the reaction reservoir 9. In embodiments that are composed of materials that are substantially IR transparent, the IR reflective surface (or coating) 10 can be positioned on the interior wall of the reaction reservoir 9, the exterior wall of the reaction reservoir 9, or within the wall of the reaction reservoir 9. In embodiments of the IR reaction vessel 1 that are composed of materials that are not substantially IR transparent (and instead have an optically transparent window), the IR reflective coating 10 can be positioned on the interior wall of the reaction reservoir 9. The IR reflective coating 10 can be composed of any suitable material, including but not limited to gold, silver, aluminum, stainless steel, platinum or any other metallic substance with good reflective property to IR.

The target absorptivity factor (at) is a measure of how well the surface absorbs the IR energy. Shiny surfaces will reflect heat away, while dull surfaces will absorb heat (most plastics are excellent absorbers of IR). Control of infrared is generally done by controlling the source temperature (Ts). The general equation for infrared heat transfer is :Q/A=Fv*es *aₜ*S*(Tₛ⁴−Tₜ⁴), where Q/A=Infrared heat transfer (W/cm2), Fv=Geometric view factor (0-1), es=Emissivity of the source (0-1), aₜ=Absorptivity of the target (0-1), s=Stefan-Boltzmann Constant, Tₛ=Source temperature (° K.), Tₜ=Target temperature (° K.). The IR reflective coating 10 effects the term “aₜ” in the equation shown above.

Certain embodiments of the present invention also include a temperature control mechanism to control the “Tₛ” factor in the equation for infrared heat transfer. In some of these embodiments, the temperature control mechanism is a closed feedback control in which the internal temperature of the reaction reservoir 9 is directly or indirectly measured. Indirect measurements can be optimized through the use of a temperature sensor sleeve mounted on the reaction vessel. A standard positioning of this temperature sensor sleeve allows reproducible placement of a temperature sensor for indirectly measuring the internal temperature of the reaction reservoir 9. In this embodiment, the temperature sensor is a thermocouple and a feedback control is featured to modulate the source temperature for a desired target temperature at the point of contact based on measurements from the thermocouple.

Alternative embodiments use an IR thermometer in a feedback control to detect the temperature directly in the reaction reservoir 9 during the heating process. The IR thermometer has the ability to focus its temperature sensing capabilities through the vessel walls and measure the solution inside the reaction vessel. Embodiments utilizing an IR thermometer do not require a thermocouple contacting the vessel surface and allow for enhanced reproducibility of product.

Still other embodiments include the mounting of a resistive thermal device (RTD) in a set location proximal to the IR reaction vessel to indirectly measure the internal temperature of the reaction reservoir 9. The RTD can be attached to a free-standing structure in air which is centered in a bisecting plane containing the long-axis of the reaction vessel. Also it can be encapsulated by a structure with similar material composition and cross-section area filled with liquid that has matching IR optical properties (attenuation, scatter, absorption, etc). This feature will minimize the difference in temperature readout of the RTD to actual temperature of solution inside the reaction vessel. In certain embodiments, the RTD is attached to a swivel arm to allow ease of reaction vessel replacement.

The reproducible placement of the IR reaction vessel with respect to the position of the IR source, or more specifically the focal point or plane of the heat transfer profile, effects the term “Fᵥ” in IR heat transfer equation. If the focal point of the heat transfer profile is not precisely consistent, major differences in heat transfer and efficiency can be observed due to the steep heat gradient of IR in both vertical and horizontal directions. Fᵥ is also critical for accurate reading on the temperature sensor and calibration. Therefore, the seating of the IR reaction vessel 1 in the support bracket 4 must be precisely reproducible.

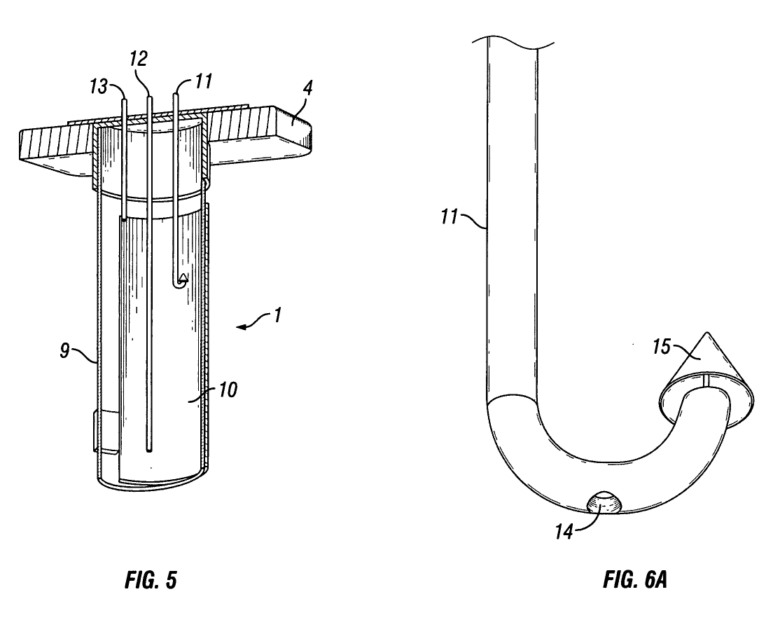

Still another feature of the present invention is the inclusion of at least one access tube crossing through the male key cap 2 into the lumen of the reaction reservoir 9. Access tubes can be composed of tight proof PEEK, Teflon, or other chemically inert material. Access tubes allow for reaction manipulations without necessitating the movement or removal of the IR reaction vessel 1. These access tubes may also be retractable prior to exposure of the infrared reaction vessel to infrared energy. Certain embodiments, such as the one shown in FIGS. 1, 4A, 4B, and 5, include one or more of the following access tubes: a vacuum tube (or line) 11, a central line 12, and/or an auxiliary line 13. When used in an automated or semi-automated radiopharmaceutical synthesis apparatus, these access lines can be connected to a closed system (such as that described in U.S. Patent Application Ser. No. 60/822,306) to decrease the possibility of contamination and ensure sterility.

In embodiments that include a central line 12, the central line can be used to simultaneously, or in tandem, deliver reactants to the reaction reservoir 9. In certain embodiments, the central line 12 can extend to position at or near the lowermost (most distal part) of the reaction reservoir 9 where it can be used to deliver or extract reagents or yields. In certain embodiments, the shape of the distal portion of the reaction reservoir 9 can aid in the collection or reactants or yields such as with the IR reaction vessel 1 depicted in FIG. 1 which has a conical bottom. Furthermore, nitrogen gas (or any other suitably inert gas) can be pumped through the central line 12 and allowed to bubble up through the reactants in the reaction reservoir to agitate, or mix, the reactants during various reaction steps in the generation of radiopharmaceuticals. In certain embodiments, the central line 12 is partially or completely removable or retractable thereby allowing the central line 12 to be removed from protruding into the reaction reservoir during certain activities, such as IR heating. In some other embodiments, a small thermocouple can also be placed inside the IR vessel via the hole in male key cap 2 for the central line 12. When the central line is replaced with a small thermocouple in this manner, the temperature calibration is performed inside the vessel versus at the outside temperature sensor.

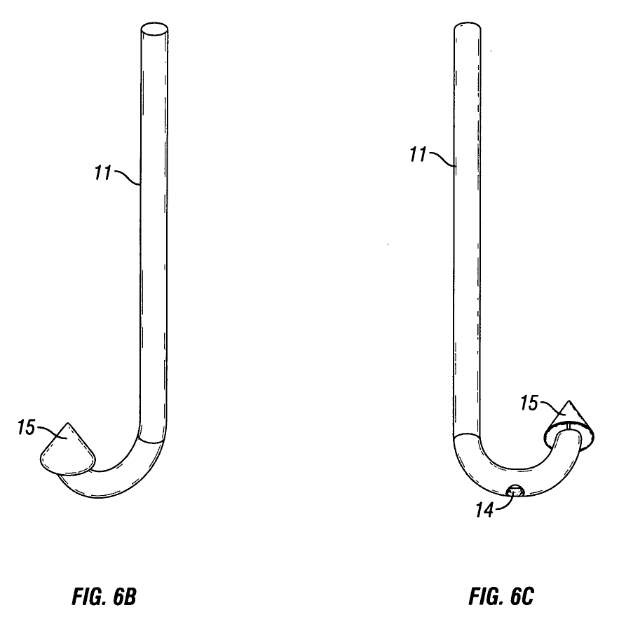

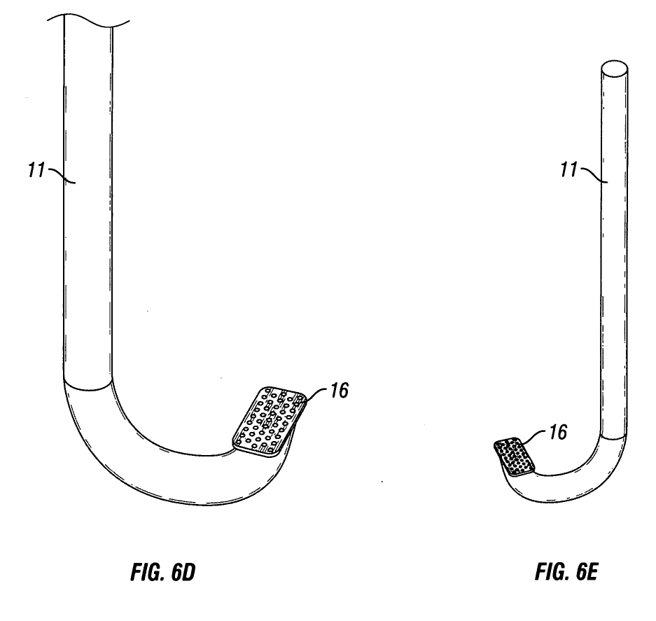

A vacuum line 11 can act as vent for gas, or vapor pressure, build-up during IR heating steps requiring a vacuum pull. In certain embodiments, the vacuum line 11 is designed to minimize radioactive loss during venting. FIG. 6A depicts an embodiment of the vacuum line 11 in a loop configuration. The reclamation port 14 acts as a reentry port for liquids taken up by the vacuum line 11 that have been condensed from a gaseous state. In certain embodiments, the vacuum line 11 can also include a condensation trap 15 at the distal end. One of ordinary skill in the art will recognize that multiple configurations are functional as a condensation trap and that the conical configuration displayed in FIG. 6A is exemplary only. For example, FIG. 6B also depicts a vacuum line 11 in a loop configuration but this embodiment includes a mesh evaporation guard 16 instead of a condensation trap 15.

In addition to an outlet tip designed aimed toward collecting or trapping condensation containing radioactivity, certain embodiments of the vacuum line 11 include an associated IR detection device placed in the reaction reservoir 9 (for example on bottom inner surface of the male key cap 2) that will activate an electronic circuit to switch on a circuit upon exposure to IR. The circuit produces electric field around the vacuum line 11 and its outlet tip to repel vaporized or gaseous ionized particles that would otherwise be evacuated from the reaction reservoir 9.

In certain embodiments, the vacuum line includes an insulated conductive wire embedded into the wall of the line to emit the electric charge field around the tubing to repel these ions. In some embodiments that include an IR detection device, a power service (or supply) 17 is included on the IR reaction vessel 1. For example, a battery can be embedded or attached to the male key cap 2 to serve as a power source 17 to provide the current to produce an electric field around the vacuum line 11.

In other embodiments, the IR reaction vessel 1 will make contact with a circuit on or in the support bracket 4 to connect the vacuum line 11 to a distal power source (i.e., a power source not located on the IR reaction vessel 1 but able to connect to it when the IR reaction vessel 1 is properly seated in the support bracket 4). In these embodiments, the high voltage source can be programmed to activate in conjunction with the IR source 5 activation without the need for an IR detection device.

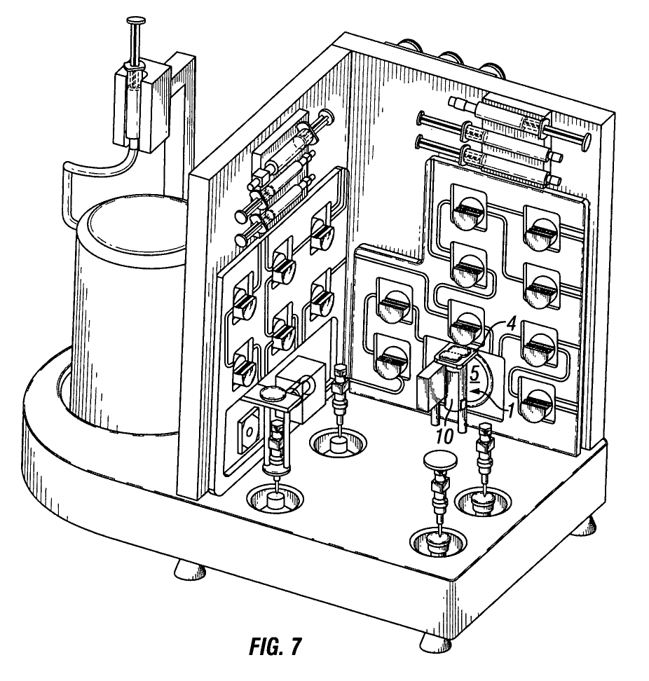

The access lines into the reaction reservoir 9 allow reactions in the IR reaction vessel 1 of the present invention to be intricately manipulated and provide convenient mechanisms for the addition and removal of reagents and products. For example, when utilized in an automated or semi-automated system such as the one depicted in FIG. 7, the central line 12 can be used to deliver a radioisotope to the reaction reservoir 9, followed by delivery of a bio-conjugate through the central line 12. The two reagents can then be agitated by delivery of nitrogen gas through the central line 12, especially in those situations in which the central line 12 extends into the liquid volume of the present reagents. In certain embodiments, the central line 12 (as well as any other access line) can be retracted from the reaction reservoir 9 prior to activation of the IR source 5 to heat the reactants and then returned to the reaction reservoir 9 after heating. As discussed above, in certain embodiments an electric current can be activated to repel vaporized or gaseous ions to minimize any loss of radioactivity through the vacuum line 11. After IR heating of the reactants, an auxiliary line 13 can be used to pump nitrogen, or any other sufficiently inert gas such as argon, into the reaction reservoir 9 thereby creating a positive pressure in the reaction reservoir 9 causing the liquid reactant and/or yield to move up out of the reaction reservoir 9 through the central line 12.

Certain embodiments of the IR reaction vessel of the present invention also allow for the concentrating of reactants and/or yields. For example, the IR heating mechanism can be used to heat and vaporize excess solvent in which a radioisotope or radiopharmaceutical (or any intermediate thereof) is suspended in conjunction with extraction of the vaporized solvent via vacuum pull through the vacuum line 11.

Furthermore, in embodiments that include an auxiliary line 13, the auxiliary line 13 can be used as an access point for delivery of reactants as well as, or in addition to, an inert gas.

Any element of the IR reaction vessel that can come in contact with any reagents and/or subsequent yields can be coated, or chemically treated, to optimize the labeling yield by minimizing the interactions between radioisotopes, radiopharmaceuticals and resulting intermediates. Such elements include but are not limited to, the inner surface of the reaction reservoir 9, the access tube(s), and the inner surface of the male key cap 2. The coating used to prevent, or retard, interactions between reactants/yields/intermediates and these surfaces can be of any suitable composition, including, but not limited to, Teflon® and silicone.

Finally, the IR reaction vessel 1 of the present invention can be made disposable with preloaded precursor reactants, solvents or prodrugs for insertion directly into the support bracket 4 thereby decreasing potential measurement errors and procedure times.

All of the apparatuses and methods disclosed and claimed herein can be made and executed without undue experimentation in light of the present disclosure. While the compositions and methods of this invention have been described in terms of embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

### EXAMPLES

### Example 1

### Synthesis of ⁶⁸Ga-DOTA-D-Phe1-Tyr3-octreotide (⁶⁸Ga-DOTATOC)

Using a protocol substantially corresponding to the conventional synthetic methodology described hereinabove (Meyer et al. 2004), an efficient IR-based reaction vessel and infrared heater of the present invention were used to synthesize ⁶⁸Ga-DOTATOC.

Prior to synthesis, a male cap containing three access tubes was attached to the reaction vessel. The access tubes were connected to 1) a vacuum line; 2) nitrogen flow; and 3) a transfer route into a final collection vial as described. The first reaction step consisted of delivering a solution of ⁶⁸GaCl₃ (in 0.1-1 N HCl) into the reaction vessel. In order to remove HCl, the heater was operated at full power until the temperature in the vessel, as measured by a temperature sensor with a feedback loop, began to rise drastically. Care was taken never to allow the temperature to rise above 120° C. to ensure minimal thermal decomposition. This step took 5-7 minutes. Dry nitrogen gas was passed through the vessel at 20 ml/min during this process, while at the same time the vessel was evacuated through a vacuum line. The infrared lamp was turned off when 1-2 liquid droplets remained in order to prevent “baking” of the ⁶⁸GaCl₃ into the glass of the reaction vessel.

The labeling step was carried out by adding 2 ml of sodium acetate (0.1 N), containing 10 μg of DOTATOC, to the radioactivity and heating at 95° C. for 5 min. The infrared lamp was operated at <50% power for this step, modulated by the temperature sensor feedback sensor. The temperature within the vessel never exceeded the boiling point of the solvent.

Following synthesis, the ⁶⁸Ga-DOTATOC solution was passed through a C-18 Sep-pak® column. The liquid eluate was collected as waste. The final ⁶⁸Ga-DOTATOC product was then eluted off the column using 2×500 μl ethanol, which was collected in a septum-sealed glass vial. By heating this in an oil bath at 90° C. under a stream of nitrogen, ethanol was evaporated within 5-6 minutes. Finally the product was dissolved in 0.9% saline, sterilized by filtration through a 0.2-μm membrane filter, and collected in a sterile vial.

The typical synthesis times for manual manipulation of liquids with this vessel averaged about 30 minutes. (This time is reduced with full automation of the process steps.) EOS (“end of synthesis”) yields are expected to be >70% owing to reduced product loss during evaporation resulting from the novel/featured vacuum line (voltage, different shapes), and refinements in the final formulation step. Radiochemical purity of the final formulation is expected to exceed 98%.

### Example 2

### Synthesis of 2-[¹⁸F]fluoro-2-Deoxy-D-Glucose (¹⁸F-FDG)

Using a protocol substantially corresponding to the conventional synthetic methodology described by Hamacher et al., Hamacher K. et al., Efficient stereospecific synthesis of NCA 2-[¹⁸F]fluoro-2-deoxy-D-glucose using aminopolyether supported nucleophilic substitution, J. Nucl. Med., 27: 235-238 (1986), herein incorporated fully by reference, an efficient IR-based reaction vessel and infrared heater of the present invention were used to synthesize ¹⁸F-FDG.

Preparation of the reaction vessel was performed substantially as described in Example 1. The first step of the radiosynthesis consists of delivering a solution of ¹⁸F (0.6 ml, 0.01 M potassium carbonate) into a reaction vessel containing 25 mg of 2,2,2-Kryptofix®. The resulting potassium fluoride/Kryptofix® complex was subsequently dried, first by distillation under vacuum and then by azeotropic distillation of the remaining water with the addition of three 0.5 ml portions of acetonitrile. During evaporation, the infrared heater was operated at full power until the temperature in the vessel, as measured by a temperature sensor with a feedback loop, began to rise drastically. Care was taken never to allow the temperature to rise above 120° C. to ensure minimal thermal decomposition.

The ¹⁸F complex was allowed to exchange with the trifluoromethylsulfonyl group on 30 mg of 1,3,4,6-tetra-O-acetyl-2-O-trifluoromethanesulfonyl-β-D-mannopyranose (triflate) which was dissolved in 2 ml of dry acetonitrile and added to the reaction vessel. This exchange occurred with the infrared lamp operating at <50% power and took 5 minutes. The solvent was then removed by distillation under vacuum.

The product (¹⁸F-triflate) was redissolved in 0.25 ml of acetonitrile and 6 ml of water and was passed onto a C-18 silica Sep-Pak®. The C-18 Sep-Pak®was subsequently washed with 4 ml of water, and 4 ml of 0.1 HCl to remove Kryptofix® from the mixture. The product was then washed off the C-18 silica Sep-Pak® to a second reaction vessel by washing twice with 2 ml portions of tetrahydrofuran.

The tetrahydrofuran was removed by distillation under vacuum, 2 ml of 2 N HCl was added, and the reaction was then allowed to reflux for 8 minutes in order to hydrolyze the acetyl protecting groups. The vessel was removed from the heat source and was partially neutralized by the addition of 1.8 ml of 2 N NaOH. Neutralization and buffering were then accomplished by the addition of 4 ml of a saturated sodium bicarbonate solution (1.2 M).

The resulting solution was passed through a neutral alumina Sep-Pak® to remove unreacted fluoride, a C-18 silica Sep-Pak® to remove organic impurities, and then passed through a 0.22 micron filter for sterilization. The final product was collected with expected yields of >60% and analyzed for sterility. In addition, High Performance Liquid Chromatography will be used to determine radiochemical purity, which is expected to be >95% for the final formulation.

What is claimed is:

1. An infrared reaction vessel system comprising:
a reaction reservoir formed by the walls of a reaction vessel;
an optical window into the reaction reservoir that absorbs minimal infrared energy;
a male cap attached to the reaction vessel; and
at least one access tube passing through the cap.

2. The infrared reaction vessel system of claim 1, further comprising an associated support bracket, wherein the support bracket remains at a fixed location relative to an infrared source.

3. The infrared reaction vessel system of claim 2, wherein the male key cap will fit into a female receptacle located on the support bracket in only one position.

4. The infrared reaction vessel system of claim 2, wherein the at least one access tube is a vacuum line.

5. The infrared reaction vessel system of claim 4, wherein reaction vessel further comprises an electric circuit attached to the vacuum line that can be activated when the reaction vessel is exposed to infrared energy.

6. The infrared reaction vessel system of claim 4, wherein the vacuum line further comprises an embedded insulated conductive wire to generate an electric field upon activation of the electric circuit.

7. The infrared reaction vessel system of claim 4, wherein the vacuum line is in a looped configuration.

8. The infrared reaction vessel system of claim 7, wherein the vacuum line further comprises a condensation trap or evaporation guard.

9. The infrared reaction vessel system of claim 4, further comprising a central line and an auxiliary line.

10. The infrared reaction vessel system of claim 9, wherein at least one of the central line, the vacuum line, or the auxiliary line is retractable prior to exposure of the infrared reaction vessel to infrared energy.

11. The infrared reaction vessel system of claim 4, wherein the reaction reservoir is substantially inert to a reaction medium.

12. The infrared reaction vessel system of claim 4, further comprising a resistive thermal device (RTD).

13. The infrared reaction vessel system of claim 12, wherein the RTD is attached at a fixed location proximal to the IR vessel.

14. An infrared reaction system comprising:
an infrared reaction vessel which includes:
a reaction reservoir formed by the walls of the reaction vessel;
an optical window into the reaction reservoir that absorbs minimal infrared energy;
a cap attached to the reaction vessel; and
at least one access tube passing through the cap, and
a support bracket for the infrared reaction vessel mounted at a fixed location relative to an infrared source.

15. The infrared reaction system of claim 14, wherein the infrared reaction vessel further comprises an infrared reflective surface attached to the reaction vessel.

16. The infrared reaction vessel of claim 14, further comprising a male key cap that will fit into a female receptacle located on the support bracket in only one position.

17. The infrared reaction vessel of claim 14, further comprising a female key cap that will fit into a male receptacle located on the support bracket in only one position.

18. The infrared reaction vessel of claim 14, wherein the at least one access tube is a vacuum line.

19. The infrared reaction vessel of claim 17, wherein reaction vessel further comprises an electric circuit attached to the vacuum line that can be activated when the reaction vessel is exposed to infrared energy.

20. The infrared reaction vessel of claim 18, wherein the vacuum line further comprises an embedded insulated conductive wire to generate an electric field up activation of the electric circuit.

21. The infrared reaction vessel of claim 17, wherein the vacuum line is in a looped configuration.

22. The infrared reaction vessel of claim 17, wherein the vacuum line further comprises a condensation trap or evaporation guard.

23. The infrared reaction vessel of claim 17, further comprising a central line and an auxiliary line.

24. The infrared reaction vessel of claim 22, wherein at least one of the central line, the vacuum line, or the auxiliary line is retractable prior to exposure of the infrared reaction vessel to infrared energy.

25. The infrared reaction vessel of claim 17, wherein the reaction reservoir is substantially inert to a reaction medium.

26. The infrared reaction vessel of claim 14, wherein the system is automated and further comprises a network of tubes in fluid communication with the at least one access tube.

27. The infrared reaction vessel of claim 14, wherein the shape of the infrared reaction vessel is matched to the heat profile generated by the infrared source.

28. The infrared reaction vessel of claim 26, wherein the cross-sectional shape of the reaction vessel is square or rectangular and the infrared source generates a strip-infrared (IR) profile.

29. A method of generating a radiopharmaceutical comprising:
providing the infrared reaction vessel system of claim 14;
delivering a radioisotope in an acid solution into the reaction reservoir through the at least one access tube;
heating the infrared reaction vessel with an infrared heater to remove at least a portion of the acid solution;
evacuating vaporized acid solution from the reaction reservoir via vacuum through at least one access tube;
delivering a labeling reagent into the reaction reservoir through at least one access tube;.
heating the radioisotope and labeling reagent in the reaction reservoir with an infrared heater.
